(19) United States
(12) Patent Application Publication
(10) Pub. No.: US 2006/0020170 A1
(43) Pub. Date: Jan. 26, 2006

(54) Appliance for use with apparatus providing therapy

(75) Inventors: Victor I. Chornenky, Minnetonka, MN (US); Ali Jaafar, Eden Prairie, MN (US)

Correspondence Address: OFFICES OF CRAIG GREGERSEN, P.O. BOX 386353, 10032 QUEBEC AVENUE SOUTH, BLOOMINGTON, MN, 55438, US

(73) Assignee: Minnesota Medical Physics LLC

(21) Appl. No.: 11/222,573

(22) Filed: Sep. 9, 2005

Related U.S. Application Data
(63) Continuation-in-part of application No. 10/938,971, filed on Sep. 10, 2004.
Application No. 10/938,971 is a continuation-in-part of application No. 10/392,167, filed on Mar. 19, 2003.

(51) Int. Cl. A61B 1/00

(52) U.S. Cl. 600/184

(57) ABSTRACT

An appliance for use in delivering x-ray radiation therapy internally at desired locations in a body is disclosed. The appliance may be inserted into the tissue to receive radiation and expanded to a desired shape, thereby providing a predetermined configuration to the tissue surrounding the appliance. The appliance can be configured to provide a spherical shape, for example, or an elliptical shape if desired.

### BACKGROUND OF THE INVENTION

The present application claims priority from U.S. patent application Ser. No. 10/392,167 (Published Application No. 20030179854), entitled “X-Ray Apparatus With Field Emission Current Stabilization And Method Of Providing X-Ray Radiation Therapy” and filed on Mar. 19, 2003 and U.S. patent application Ser. No. 10/938,971 (Published Application No. 20050038488), also entitled “X-Ray Apparatus With Field Emission Current Stabilization And Method Of Providing X-Ray Radiation Therapy” and filed on Sep. 10, 2004.

The present invention relates generally to an appliance useful in aiding apparatus and methods providing x-ray radiation therapy and specifically to such apparatus and methods for providing x-ray radiation therapy with real-time stabilization of the operating current, and thus the dosage rate. Without limitation to the foregoing referenced patent applications, those applications describe apparatus and method providing x-ray radiation therapy generated by a field effect x-ray emission device. The configuration and unique features of those inventions enable a therapist to provide radiation therapy at an internal location in a body.

In using x-rays for medical therapy it is important that the proper dose rate be applied. The dose depends upon the energy of the x-rays and the intensity of the x-ray beam. In field emission devices, increasing the voltage of the electric field increases the energy of the x-rays while increasing the current increases the intensity of the beam. Higher energy x-rays penetrate to greater depths in body tissue, so voltage control is important in controlling the energy to avoid damaging healthy tissue needlessly due to an undesired depth of penetration of the x-rays. The beam flux is also dependent upon the gap between the anode and the cathode. Increasing the gap decreases the beam flux and vice versa.

An undesirable feature of known field x-ray emitter devices is the inability to closely control the dose rate. One reason for this lack of control is that the generation of the electron beam from the cathode can be sporadic. That is, due to uncontrollable changes in the condition of the electron emitting surface of the cathode, field emitters are known for instability of their current, which can vary by a factor of 2. At the higher end of this range the emission current can overheat the emission site and create a vacuum discharge over the gap that can significantly change the electric properties of the gap or even make the device inoperable in the required settings. Because of the inconsistency in the current and thus the x-ray beam flux, the dose applied during any particular therapy session may not be well known, which leads to inconsistent treatment and results. The only sure way to know that a particular medical problem has been adequately addressed is to apply radiation at a presupposed rate that increases the likelihood of damage to healthy tissue.

The inventions disclosed and claimed in the priority applications referenced above disclose apparatus that enables an operator of an x-ray apparatus to control the energy and intensity of an emitted x-ray beam by independently controlling the voltage and operating current, respectively. Generating x-rays with the inventions disclosed therein will enable to control the dose rate applied to tissues.

Generally, when excising cancerous tissue, such as breast cancer, the surgeon will remove the tissue as well as a surround margin of tissue that may be healthy. To ensure that all of the cancer cells have been killed, radiation is provided. Due to the perhaps irregular configuration of the cancerous tissue and the removed margin tissue, providing a controlled dose of therapeutic radiation to the remaining, surrounding tissue can be problematic, resulting is some tissue receiving too much radiation and being injured and some tissue perhaps not receiving enough to kill any remaining cancer cells.

Thus, it would be desirable to have an appliance that could be used with such field emission apparatus to provide a desired tissue cavity configuration into which the field emission apparatus could be deployed. This would ensure that the proper dose of radiation would be provided to the tissue surrounding the cavity and to the desired depth

### BRIEF DESCRIPTION OF THE INVENTION

The present invention provides an appliance for use in delivering x-ray radiation therapy internally at desired locations in a body. Broadly speaking, the appliance may be inserted into the tissue to receive radiation and expanded to a desired shape, thereby providing a predetermined configuration to the tissue surrounding the appliance. The appliance can be configured to provide a spherical shape, for example, or an elliptical shape if desired.

In accordance with the invention, an appliance may have a bulb that is expandable and contractible between at least the extremes of expansion and contraction, though intermediate states therebetween can also be provided as desired. The bulb will preferably be formed from a predetermined number of spokes extending between a bulb base and a bulb collar such that movement of the bulb base and bulb collar relative to each other will cause the spokes to either flex outwardly to an expanded position or be pulled inwardly into a contracted position. The bulb may be configured such that the expanded bulb can assume a substantially spherical shape or a substantially elliptical shape as desired.

The present invention may further include a bulb member having a bulb member base that is attachable, either removably or permanently, to the bulb base such that the bulb member extends through and out of the bulb. The bulb member may have a central passage for receiving an x-ray probe of the type described in the aforementioned incorporated patent applications. A latch may be provided that extends and operates between the bulb member and the bulb to latch the bulb into the desired position of expansion or contraction.

In one embodiment of the invention, the bulb may include a collar extension having a central passage configured to receive the bulb member. A latch useful with such an embodiment may provide a knob that is attached to the bulb, in particular the bulb collar, that is inwardly extending, and that is received by one of a plurality of appropriately configured holes in the bulb member. To engage and disengage the knob from the holes, the collar extension may include a substantially saw-tooth shaped ramp surface that engages the inner surface of the collar. Relative motion of the collar and the collar extension will cause the collar to move inwardly and outwardly with respect to the collar extension and thus the bulb member, thereby causing the knob to engage and disengage the holes.

In another embodiment of the present invention latch may be provided by a latch button that is attached to or integrally part of the bulb member.

In another embodiment of the invention, the latch may be provided by a latch bar that is attached to the bulb member. The latch bar may have a finger rest such that pushing on the finger rest pushes the latch pin out of one of a plurality of appropriately configured latch bar grooves disposed on the collar extension, thus freeing the collar extension, and hence the collar, for relative motion relative to the bulb base for expanding and contracting the bulb.

To aid in the utilization of the appliance, a pair of grips in the form of finger rings or finger rests may be provided that engage the bulb member at the end thereof.

It will be understood that the various figures included and described herein are of various scales to enable the various features of the present invention to be shown more clearly.

The present invention, as well as its various features and advantages, will become evident to those skilled in the art when the following description of the invention is read in conjunction with the accompanying drawings as briefly described below and the appended claims. Throughout the drawings, like numerals refer to similar or identical parts.

### BRIEF DESCRIPTION OF THE DRAWING

FIG. 1 shows an embodiment of the present invention in a perspective view.

FIG. 2 shows the bulb of FIG. 1 in a perspective view.

FIG. 3 illustrates the “top” portion of a bulb that is manufactured in two parts for later assembly together.

FIG. 4 depicts the “bottom” portion of a bulb that is manufactured in two parts for later assembly together.

FIG. 5 shows the bulb member used with the appliance shown in FIG. 1.

FIG. 6 shows a perspective view of the bulb member of FIG. 5 and illustrates in particular the central passage therein for receiving an x-ray radiation therapy device.

FIG. 7 depicts in a perspective view the collar extension of the appliance shown in FIG. 1.

FIG. 8 illustrates bulb member and the collar extension in the manner in which they interact.

FIG. 9 illustrates the bulb member of FIG. 1 and its association with the finger rings shown in the appliance of FIG. 1.

FIG. 10 illustrates one of the two finger rings shown in FIG. 1.

FIG. 11 illustrates the other of the two finger rings shown in FIG. 1.

FIG. 12 depicts a top plan view of the bulb with the bulb member shown in phantom cross-section.

FIG. 13 illustrates an alternative embodiment of the present invention.

FIG. 14 illustrates a bulb member useful with the embodiment shown in FIG. 13.

FIG. 15 depicts another embodiment 102 of an appliance in accord with the present invention.

FIG. 16 shows the appliance of FIG. 15 in another view.

FIG. 17 illustrates a top bulb half in a perspective view.

FIG. 18 shows a collar extension useful with the embodiment shown in FIG. 15.

FIGS. 19A and 19B illustrate a cantilevered latch member useful with the embodiment shown in FIG. 15.

FIG. 20 shows a cross-sectional view of the embodiment shown in FIG. 15.

FIG. 21 shows a flexible sleeve that may be used with the present invention.

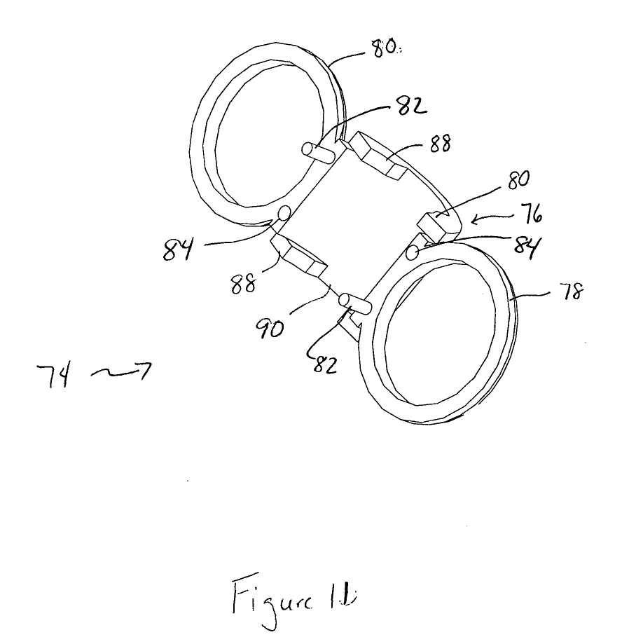

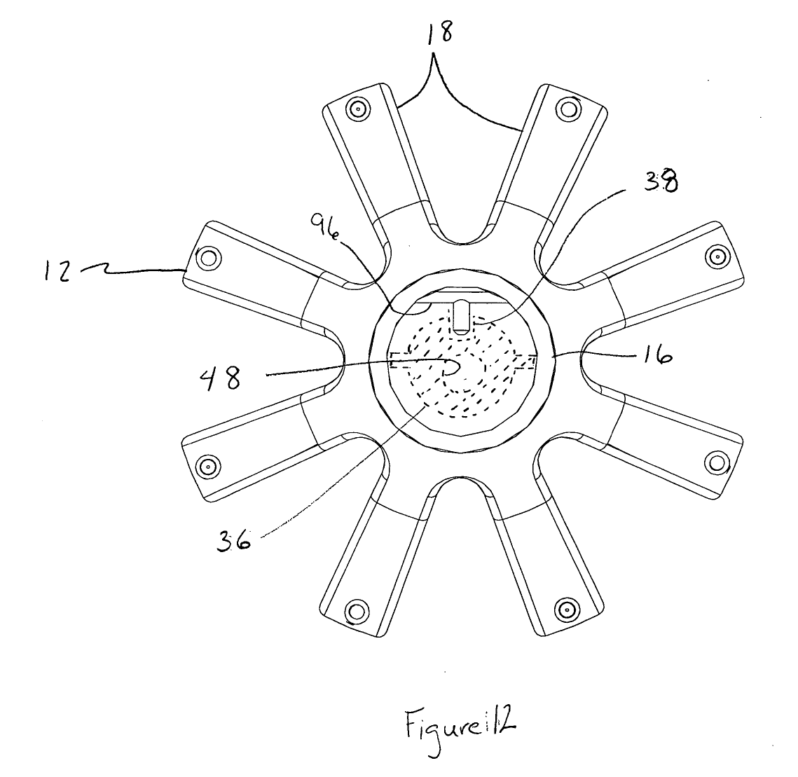

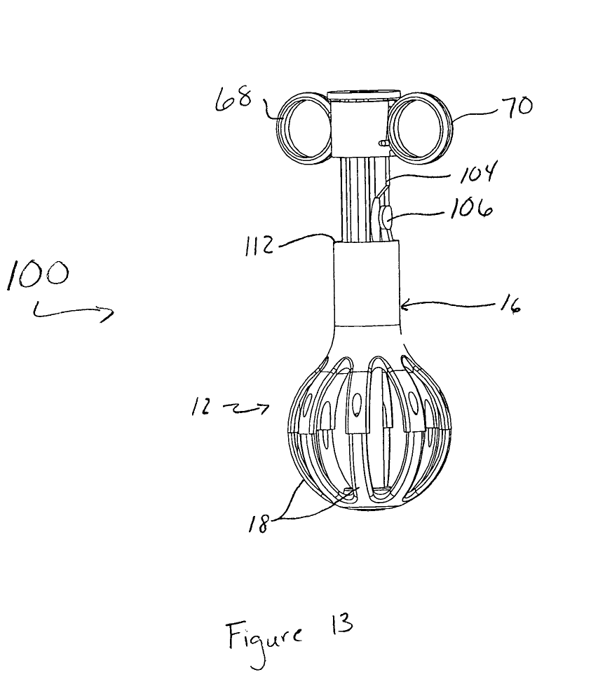

FIG. 13 illustrates the embodiments of FIGS. 11 and 12 in use with the present invention intra-operatively in a body cavity.

### DETAILED DESCRIPTION OF THE INVENTION

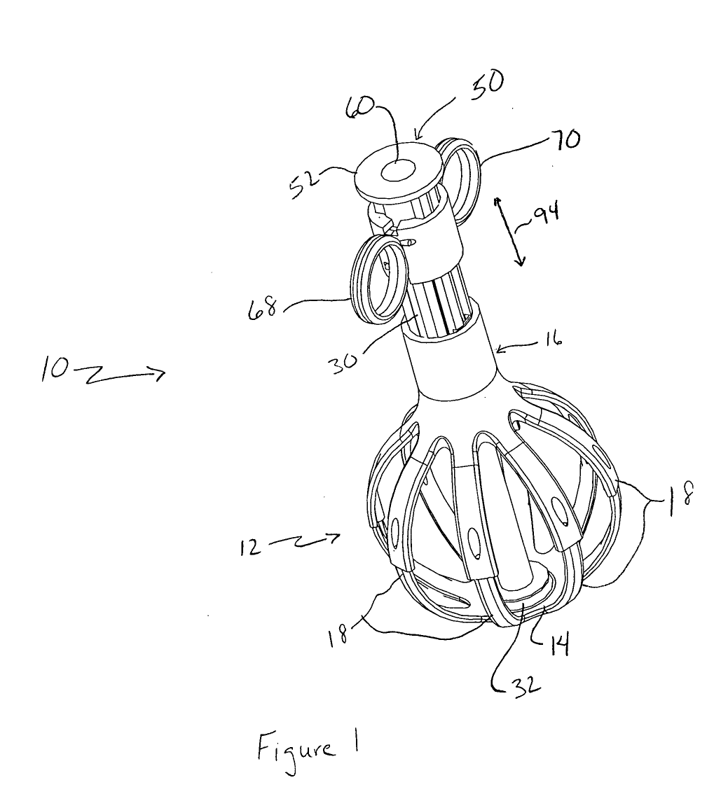

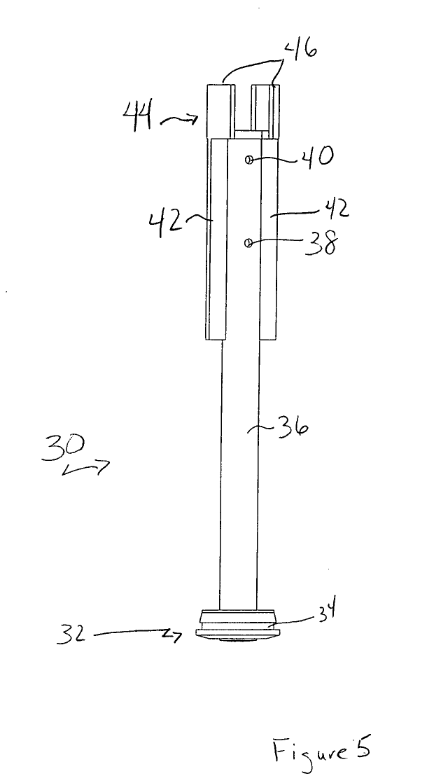

Referring now to FIGS. 1-10, an embodiment of the present invention will now be described. Thus, as shown in the Figures, an appliance 10 may include an expandable/contractible bulb 12 including a bulb base 14 and a collar 16. Bulb 12 may include a plurality of spokes 18 extending between the bulb base 14 and collar 16. The particular embodiment shown in FIGS. 1-10 includes 8 such spokes, though other numbers of spokes may be used as desired and appropriate for the tissue in which the appliance 10 may be used.

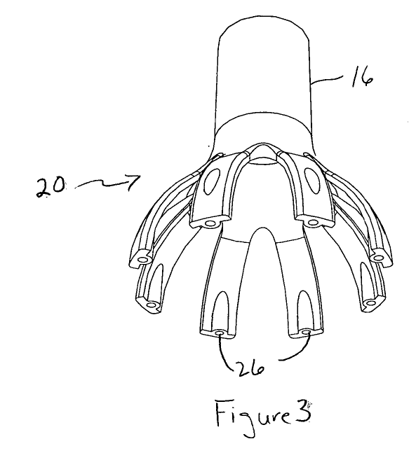

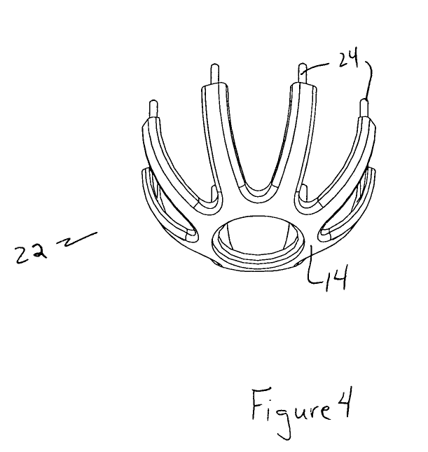

The bulb 12 may be manufactured as a single integral item, or it may be manufactured in parts and assembled. For example, as best seen in FIGS. 3 and 4, the bulb 12 may comprise a top bulb half 20 including collar 16 and a bottom bulb half 22. Following manufacture the two halves 20 and 22 may be attached to each other in any known manner appropriate for its intended use. For example, in the embodiment shown in the Figures, each partial spoke 18 in the bottom bulb half 20 includes a crush pin 24 that is configured to be received by the appropriately configured pin receiving hole 26 in the corresponding partial spoke in the top bulb half 20. The attachment of the two halves 20 and 22 can be made secure by heat staking or sonic welding or any other desirable form of attachment.

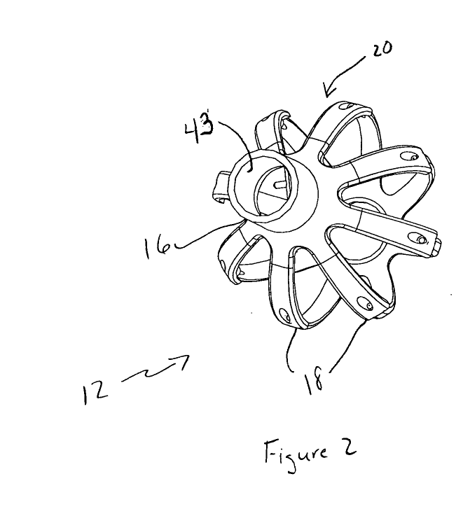

FIGS. 1, 5 and 6 show a bulb member 30 including a bulb member base 32 that is attached to the bulb base 14. The bulb member base 32 includes a circumferentially extending groove 34 configured to receive the inner mating edge 36 of the bulb base 24, best seen in FIG. 2. Preferably the diameter of the edge 36 will be slightly less than that of the groove 34 such that the bulb member base 32 “snaps” into position in the bulb base 14 and is held there tightly. If desired, the bulb member 32 may be permanently attached to the bulb base 14 in any known manner or may be manufactured as an integral unit therewith.

The bulb member 30 further includes an elongate tubular portion 36 that extends upwardly from the bulb 12. Tubular portion 36 includes at least a pair of positioning holes 38, 40 that is used to position the bulb 12 in the desired position of expansion or contraction as will be described in greater detail later. As stated, a pair of such positioning holes are shown, though more could be provided if desired. The tubular portion 36 further includes a pair of ribs 42 extending outwardly on opposite sides therefrom. Ribs 42 bear against the inside surface 43 (FIG. 2) of the collar 16 and aid in maintaining the position of the bulb member 30 along the central longitudinal axis of the bulb 12. Stated otherwise, the bulb member 30 defines the longitudinal axis of the appliance 10.

Bulb member 30 may also comprise a fixture 44 at the end thereof. Fixture 44 as shown includes a pair of opposed substantially annularly configured ear members 46. Annular ear members 46 may be used to secure appropriate finger/hand grips to appliance 10 as will be described below.

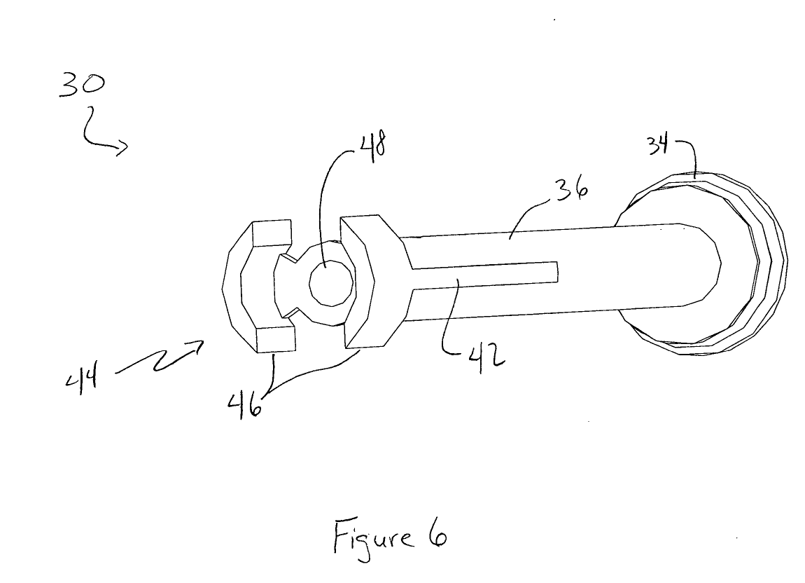

Bulb member 30 also includes a centrally disposed passage 48 (FIG. 6) that is appropriately sized to receive an x-ray radiation therapy device. As noted earlier, the positioning ribs 42 function to align the bulb member along a precise longitudinal axis. Thus, disposing the therapy device within passage 48 enables the therapist to know the precise location of the therapy device for radiation therapy.

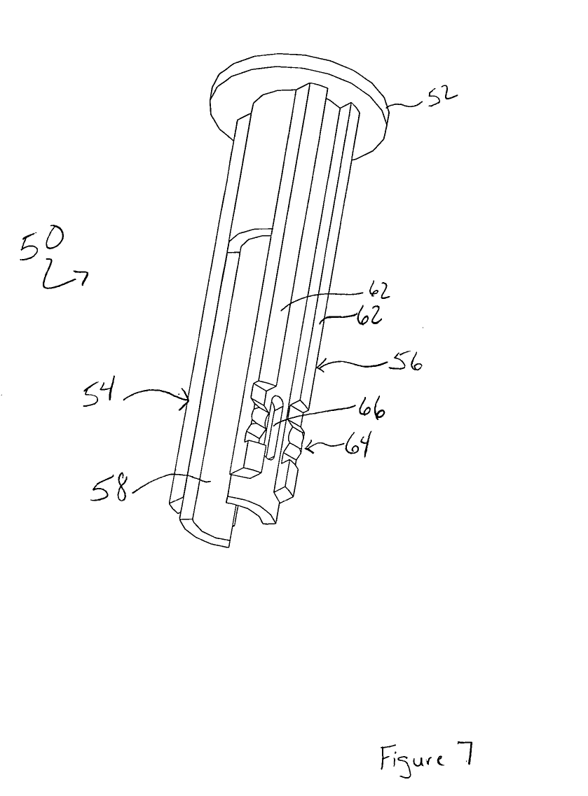

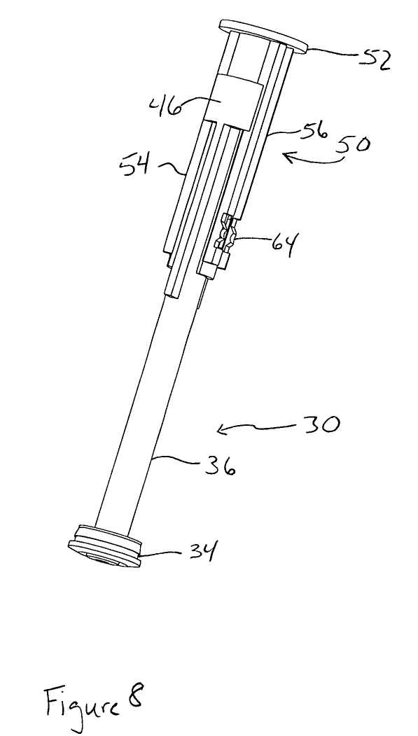

FIG. 7 illustrates a collar extension 50 useful with appliance 10. Collar extension 50 includes a cap 52 and a pair of opposed downwardly extending arms 54 and 56. The arms are spaced apart to define a channel 58 configured to slidably receive the bulb member 30 as seen in FIG. 8. As seen in FIG. 1, the collar extension 50 includes a collar extension passage 60 passing centrally therethrough. In the fully assembled apparatus shown in FIG. 1, the passage 60 and the bulb member passage 48 are aligned so as to permit the insertion of an x-ray radiation therapy device therein.

Referring to FIG. 7 again, collar extension arm 56 includes a pair of spaced apart ribs 62 each having a substantially saw-tooth shaped ramp 64 on the surface thereof. The ramp 64 includes a plurality of hills/valleys arranged sequentially that bear against the collar 16. Disposed therebetween on the arm 56 is an elongate slot 66 that extends through the arm 56. As will be discussed below, the ramps 64 and slot 66 aid in the positioning of the bulb 12 in the desired position of expansion or contraction.

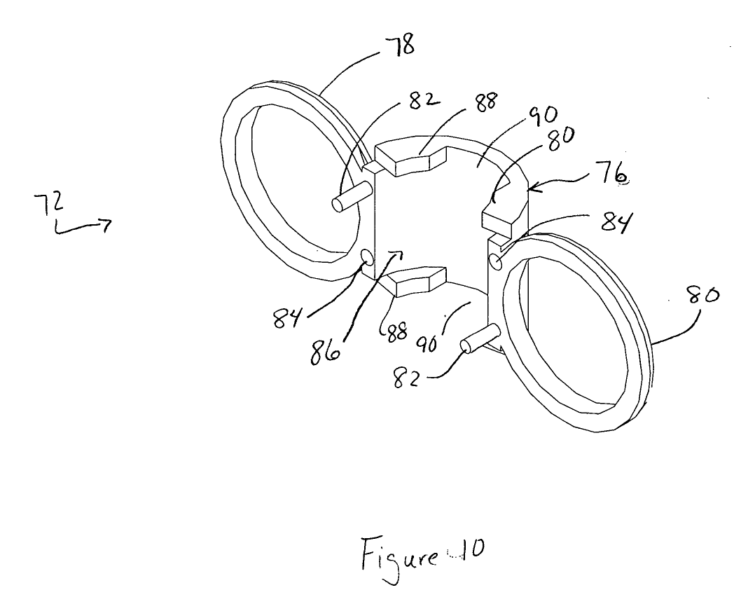

Referring now to FIGS. 1 and 9-11, the appliance 10 may include finger rings 68 and 70 to aid in the operator's use of the apparatus. Finger rings 68 and 70 may be formed by a pair of finger ring portions 72 and 74 shown in FIGS. 10 and 11. As seen there, each portion 72 and 74 includes a substantially half-cylindrically configured fixture capture portion 76 with attached finger ring members 78 and 80. When portions 72 and 74 are attached to each other, the finger ring member 76 of one portion will cooperate with the finger ring member 78 of the other portion to form one of the finger rings 68 or 70 as seen in FIG. 1. The finger ring portions 72 and 74 may be joined together through the use of pegs 82 and corresponding appropriately configured peg-receiving holes 84.

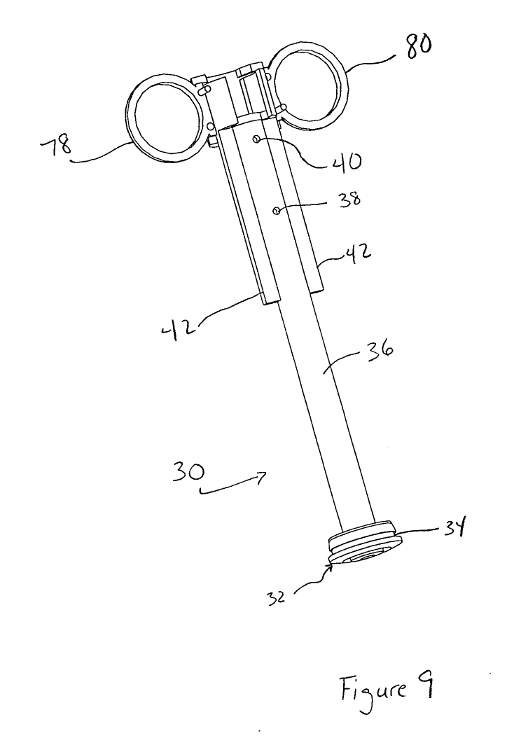

Fixture capture portions 76 of finger ring portions 72 and 74 cooperate to form a fixture receiving chamber having substantially cylindrically shaped configuration. As best seen in FIGS. 1 and 9, the fixture 44 is received within the chamber formed when finger ring portions 72 and 74 are joined together. Each fixture capture portion 76 includes a substantially cylindrical half-wall 86. Extending inwardly from the half-walls 86 are top wall segments 88 that define therebetween a “cut-out” portion 90 that receives an arm 54 or 56 of the collar extension 50 as best seen in FIG. 1.

FIG. 12 illustrates the bulb 12 in a top plan view and its interaction with the bulb member 30, shown in phantom cross-section. It will be observed that the collar 16 of the bulb 12 includes a positioning pin 92 that is received within one of the positioning holes 38 or 40. The positioning pin 92 holds the bulb 12 in the desired position of expansion or contraction.

Having described the appliance 10, its operation will be described. To expand and contract the bulb 12, the operator/therapist will grasp the appliance 10 with one hand using the finger rings 68 79 and with the other hand will grasp the cap 60 of collar extension 50 and move it upwardly or downwardly as indicated by double-headed arrow 94 in FIG. 1. As the collar extension 50 moves, the ramp 64 will bear against a ramp-bearing surface 96 (FIG. 12). As the ramp 64 moves against the surface 96, the various hills and valleys of the ramp 64 will travel against the surface 96. As a “hill” is encountered by the ramp-bearing surface 96, the collar 16 in the vicinity of the positioning pin 92 will be forced outwardly, thus pulling the positioning pin 92 out of a positioning hole, such as hole 38 or 40. Encountering a “valley” will allow the collar 16 to relax inwardly and setting the pin in the desired positioning hole.

The pin 92 and the positioning holes such as holes 38 and 40 thus act as a latch to latch the bulb in the desired state of expansion or contraction. In use, the bulb 12 will be contracted for insertion into the desired position in the body and then expanded. A therapeutic x-ray apparatus as shown in the applications referenced above may be inserted into the passages 48 and 60 of the bulb member 30 and collar extension 50, respectively, and x-ray therapy may be provided at the desired location.

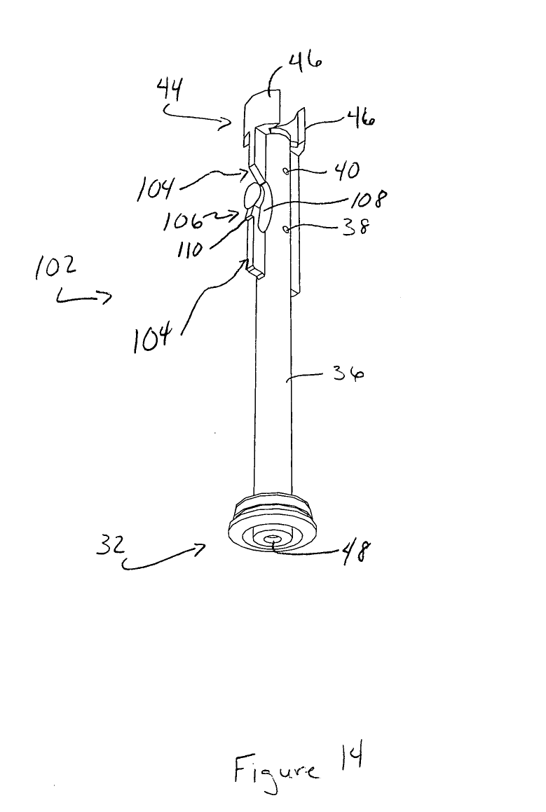

Referring to FIG. 13 another embodiment 100 of an appliance in accord with the present invention is shown. Appliance 100 includes a bulb 12, collar extension 50 and finger rings as previously described. FIG. 14 shows a bulb member 102 useful with the appliance 100. As shown there, the bulb member 100 includes a bulb member base 32, a tubular portion 36, positioning holes 38 and 40 and a passage 48, fixture 44 with opposed substantially annularly configured members 46, and a rib 42, all as previously described with respect to bulb member 30. Bulb member 102 further includes a rib 104 on the opposite side of portion 36 from rib 42. Rib 102 as best seen in FIG. 14 includes compressible latch button 106 formed in part by removing a portion of the rib 104 to create a gap 108 between the button 106 and the tubular portion 36. Gap 108 allows button 106 to be pressed backwards toward the tubular portion 36. The latch button 106 includes an outwardly projecting hook 110.

Referring to FIG. 13, it will be observed that the latch button 106 is depressed backwardly such that the hook 110 is hidden within the collar 16. By moving the collar 16 downwardly relative to the bulb base 14, the bulb will expand, the hook 110 will be exposed, and the latch button will move outwardly to its natural position. The hook 110 will catch on the upper edge 112 of the collar 16 and hold the bulb in position. That is, the hook will prevent the bulb 12 from contracting by retaining the collar 16 in position.

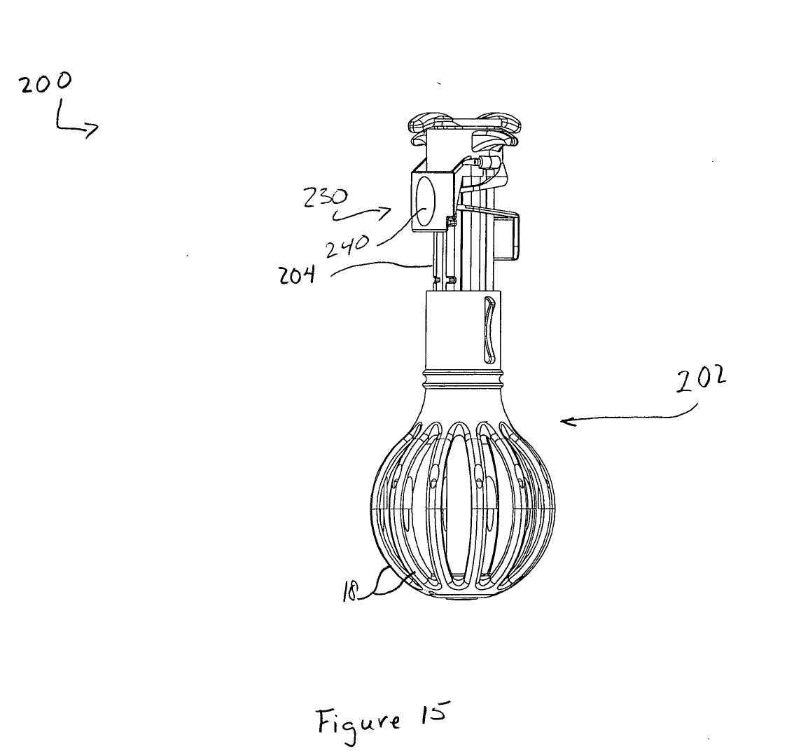

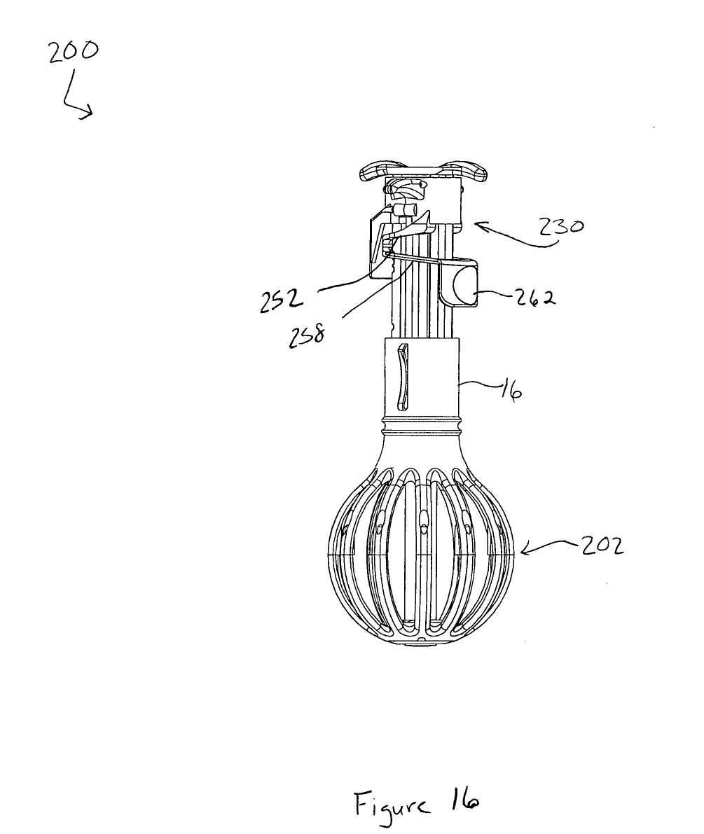

Another embodiment 200 of the present invention is shown in FIGS. 15-20. Appliance 200 may include a bulb 202 similar to that previously described, a bulb member 30 as previously described, a collar extension 204, as well as other features described hereafter.

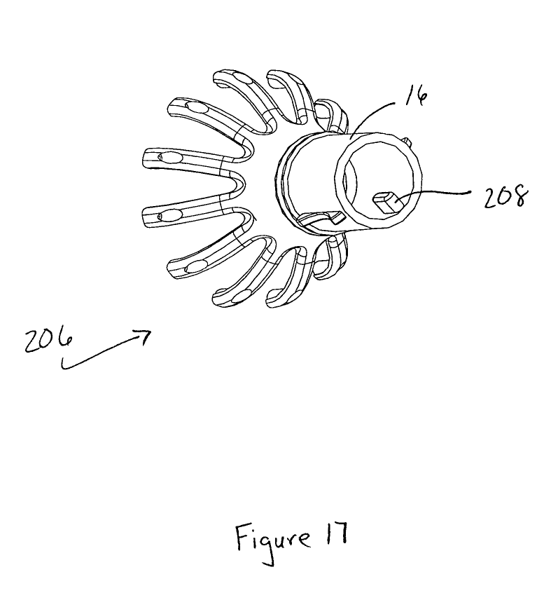

Referring to FIG. 17, a top half 206 of bulb 202 is shown in a perspective view looking down the collar 16 into the bulb. Extending inwardly from the inner surface of the collar 16 is an attachment stud 208, which will be used to attach the collar extension 204 to the collar 16 as described further below.

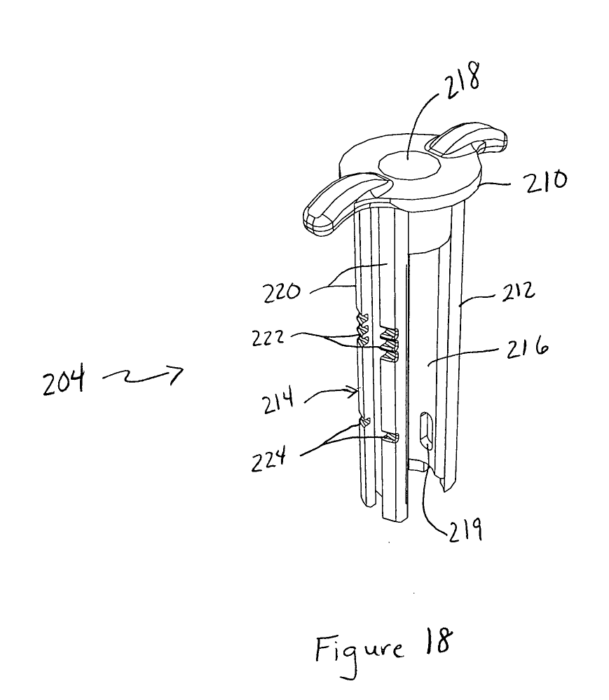

The collar extension 204 is shown in FIG. 18. As with the previously described collar extension 50, collar extension 204 includes a cap 210 and a pair of opposed downwardly extending arms 212 and 214. The arms 212 and 214 are spaced apart to define a channel 216 configured to slidably receive the bulb member 30 as seen in FIGS. 15-16 and 20 As seen in FIG. 18, the collar extension 204 includes a collar extension passage 218 passing centrally therethrough. In the fully assembled apparatus shown in FIG. 15, the passage 218 and the bulb member passage 48 are aligned so as to permit the insertion of an x-ray radiation therapy device therein.

Referring to FIG. 18 again, collar extension arm 212 includes an aperture 219 therethrough configured to receive stud 208 and hold the collar extension 204 in position relative to the collar 16.

Also seen in the Figure are a pair of spaced apart ribs 220 each having a plurality of grooves 222 therein that aid in the positioning of the bulb 12 in the desired position of expansion or contraction. Spaced apart from the plurality of grooves 222 further down the ribs 220 is a solitary groove 224 that marks the greatest contraction of the bulb 202 whereas the grooves 222 mark a plurality of degrees of expansion of the bulb.

Appliance 200 is assembled substantially identically to the previous embodiments, with the bulb member 30 being attached to the bulb base and extending upwardly and outwardly therefrom. The passage 216 of collar extension 204 receives the bulb member 30.

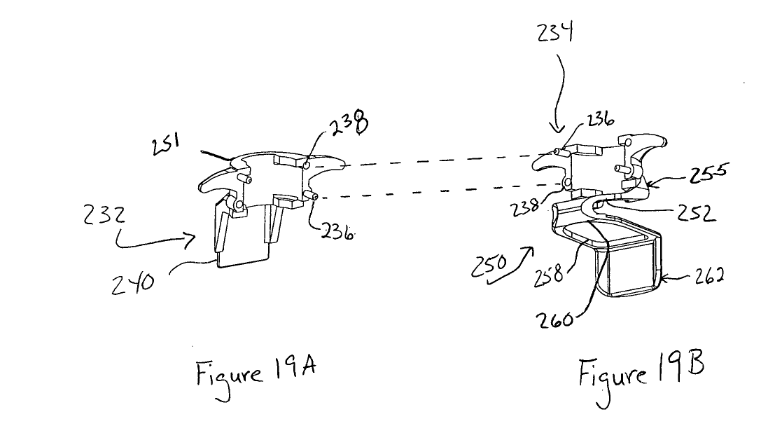

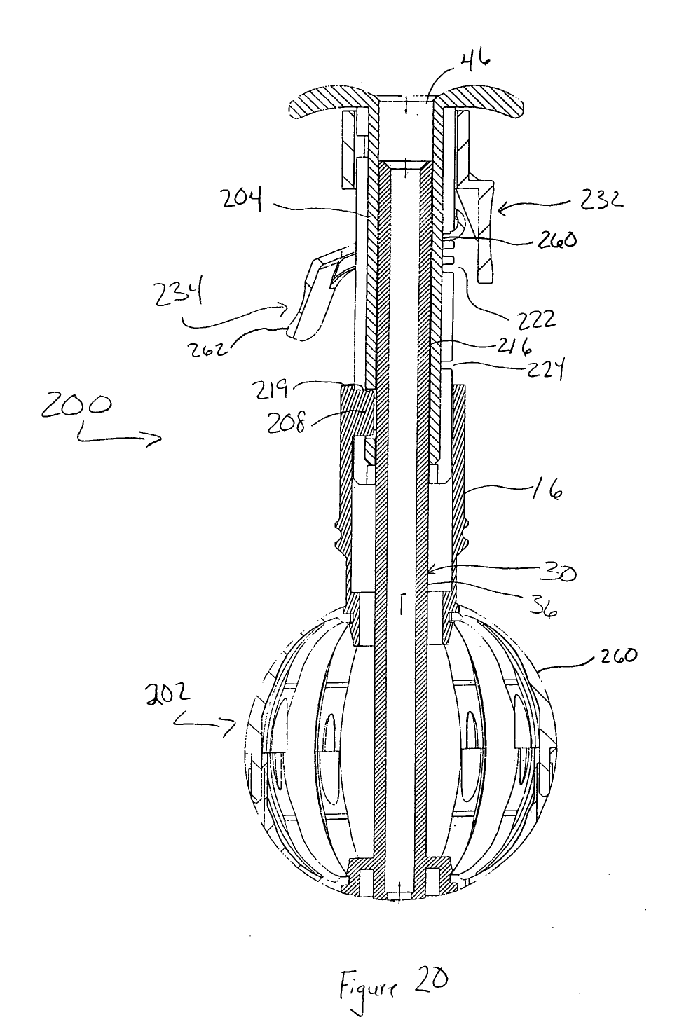

Also shown in the Figures an particularly in FIGS. 19A and 19B are grip and latch bar 230 comprising finger rest portion 232 and latch bar portion 234. Portions 232 and 234 are attached to each other using complimentary pegs and holes 236 and 238, respectively, similar to the pegs and holes 82 and 84, respectively, as previously described. When attached, portions 232 and 234 define a chamber that receives the fixture 44 of bulb member 30.

Finger rest portion 232 includes a finger rest 240, which as shown is downwardly depending, and a fixture capture portion 241.

Latch bar portion 234 includes a lever portion 250 depending downwardly. As depicted, the lever portion 250 includes a first somewhat U-shaped portion 252 and a second somewhat U-shaped portion 254. As best seen in FIGS. 15-16 and 20, first portion 252 is attached to the fixture capture portion 255 of latch bar portion 234 and defines a passage 256 through which the bulb member 30 and collar extension 204 pass. Second portion 254 is attached to the first portion 252 and also defines a passage 258 through which the bulb member 30 and collar extension 204 pass. Where the first and second portions 252 and 254 are attached to each other, a latch bar 260 is created that will engage the grooves 222, 224. The latch bar portion 234 may also include a finger rest 262. When assembled, the fixture capture portions 251 and 255 will form a substantially cylindrical chamber that will capture the fixture 44 of the bulb member 30.

Referring to FIGS. 15, 16 and 20 in particular, the operation of the embodiment 200 will be explained. As seen in those figures, pressing on the finger rest 262 will cause the latch bar 260 to pivot out of engagement with one of the grooves 222, 224 (whichever groove it is in at that point in time). Once disengaged, the bulb member 30 can be moved reciprocally within the channel 216 in the collar extension 204 to the desired position of expansion or contraction of the bulb. When correctly positioned, the pressure on the finger rest 262 can be released to engage the latch bar 260 in a groove 222, 224 as desired.

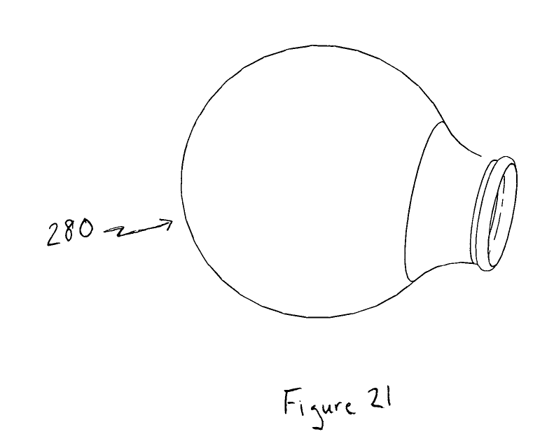

FIG. 20 is a cross-sectional illustration of the embodiment shown in FIG. 15. It will be observed that this illustration shows that the collar 16 can be manufactured as a separate part apart from the top portion 270 of the bulb 202. When so manufactured the collar 16 and top portion 270 can be attached by any known means, including the slot and groove attachment shown in the Figure.

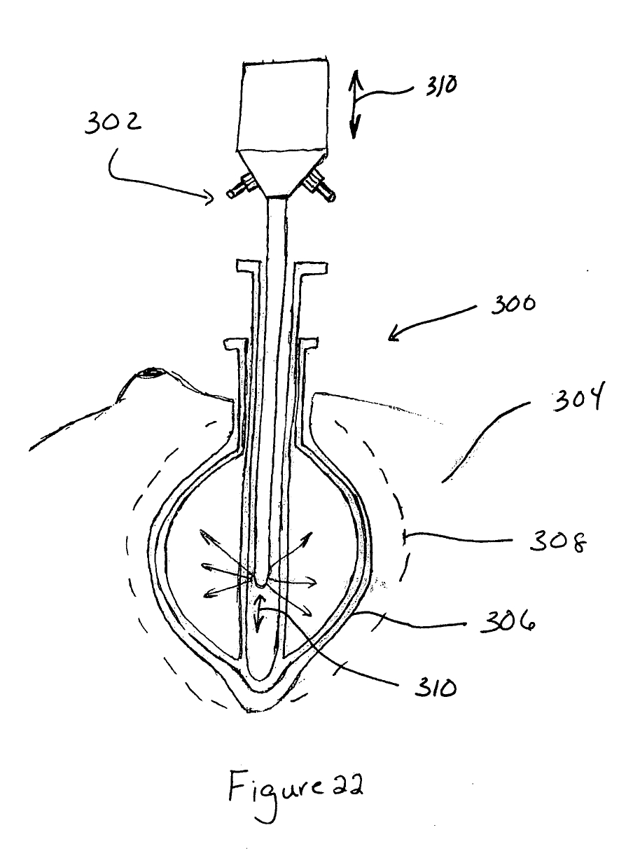

FIG. 21 shows a compliant or flexible skin 280 that may be secured over the bulb 12 of either of the embodiments shown herein. If desired, the skin 280 may be hermetically sealed to the bulb 12, thereby providing a solid surface to the tissue receiving therapy.

FIG. 22 provides a highly schematic illustration of an example of the use of an appliance 300 of the type shown in FIGS. 1-20 in combination with an x-ray apparatus 302 as described in the referenced and incorporated patent applications for an irradiation treatment following surgical excision of a breast tumor. Thus the Figure shows a breast 304 having a cavity 306 created by the excision of a tumor. The cavity 306 has been expanded by the use of the appliance 300 as previously described. A target zone for depth of irradiation of the margin tissue surrounding the tumor is indicated by the dotted line 308. As indicated by double headed arrow 310, the apparatus 302 is movable back and forth relative to the cavity 306, facilitating irradiation of the target zone. As noted earlier, the appliance 300 would be inserted into the cavity 306 and would then be placed into the expanded or tissue stretching configuration shown in FIG. 22. If desired, the appliance can be inserted alone and then the apparatus 302 may be inserted therein, or the apparatus 302 may first be placed with the appliance and the appliance then inserted. The use in the Figure contemplates the use of the appliance disclosed herein.

At least the spokes and bulb member of the present invention should be relatively transparent to x-ray radiation. Stated otherwise, they should be manufactured of a material have a low x-ray absorption or at least equal to and preferably less than that of soft tissue. Such materials include, among many others known to those skilled in the art, low density polyethylene, foamed plastic, polycarbonate impregnated plastics. Any material that provides low radiation absorption and that provides the functions specified herein, such as plastics and metals, may be used.

The present invention has been described in language more or less specific as to the apparatus and method features illustrated in the Figures. It is to be understood, however, that the present invention is not limited to the specific features described, since the apparatus and method herein disclosed comprise exemplary forms of putting the present invention into effect. The invention is, therefore, claimed in any of its forms or modifications within the proper scope of the appended claims appropriately interpreted in accordance with the doctrine of equivalency and other applicable judicial doctrines.

What is claimed is:

1. An appliance for expanding a cavity in tissue, the appliance comprising:
a bulb expandable and contractible at least between fully expanded and fully contracted positions, the bulb having distal and proximal bulb ends, the proximal bulb end including a collar having a substantially circular cross-section, wherein the bulb includes a plurality of spokes extending between the distal bulb end and the proximal bulb end;
a bulb member having proximal and distal support member ends, wherein the bulb member includes a centrally disposed passage for receiving a therapeutic device therein and is configured to be received within the bulb and the distal support member end is attached to the distal bulb end; and
a latch operating between the bulb member and the collar to latch the bulb in a desired position of expansion or contraction,
wherein the bulb can be disposed in its contracted position, inserted into tissue where a cavity is desired, expanded to an expanded position, and latched in position.

2. The appliance of claim 1 wherein at least the spokes and bulb member are made from a material having low x-ray absorption

3. The appliance of claim 1 wherein at least the spokes and bulb member are made of a material selected from on of the following: low density polyethylene, foamed plastic, or polycarbonate impregnated plastic.

4. The appliance of claim 1 and further including a flexible sleeve disposed over the spokes to provide a solid interface to the tissue.

5. The appliance of claim 1 wherein the collar includes a collar extension attached to and extending away from the bulb and wherein the latch operates between the bulb member and the collar extension.

6. The appliance of claim 5 wherein the collar extension is integrally attached to the collar.

7. The appliance of claim 5 wherein the latch comprises a plurality of grooves disposed in the surface of the collar extension and a latch bar pivotally attached to the bulb member such that the latch bar may be pivotally moved into and out of engagement with the grooves.

8. The appliance of claim 1 wherein the latch comprises a positioning pin attached to the collar and a positioning pin receiving hole disposed on the bulb member.

9. The appliance of claim 9 wherein the collar includes a collar extension attached to and extending away from the bulb.

10. The appliance of claim 8 wherein the collar extension includes a substantially saw-tooth shaped ramp and the collar includes a ramp bearing surface and wherein movement of the collar extension relative to the collar causes the ramp to engage the ramp bearing surface and to alternately move the collar outward such that the positioning pin disengages from a positioning pin receiving hole.

11. The appliance of claim 1 wherein the latch comprises a latch button having a hook thereon, the latch button being attached to the bulb member such that it is pivotal towards and away from the bulb member and wherein the latch button latches the appliance into its expanded position by latching onto the collar.

12. The appliance of claim 1 wherein at least the spokes and bulb member are made of plastic.

13. The appliance of claim 1 wherein at least the spokes and bulb member are made of metal.
